(19) United States
(12) Patent Application Publication
(10) Pub. No.: US 2019/0381305 A1
(43) Pub. Date: Dec. 19, 2019

(54) Locking Bag Forming Peripherally Inserted Central Catheter (PICC) Guard

(71) Applicant: Jesse Michael Justus, Sheboygan, WI (US)

(72) Inventor: Jesse Michael Justus, Sheboygan, WI (US)

(21) Appl. No.: 16/243,860

(22) Filed: Jan. 9, 2019

Related U.S. Application Data
(60) Provisional application No. 62/615,173, filed on Jan. 9, 2018.

(51) Int. Cl. A61M 39/10; A61M 25/02

(52) U.S. Cl. CPC A61M 39/1011; A61M 25/02; A61J 1/1475; A61M 2039/1066; A61M 2039/1077

(57) ABSTRACT

A puncture resistant bag has an open end into which is inserted the injection port of a Peripherally Inserted Central Catheter (PICC) assembly which has a flexible catheter. The open end of the bag is irreversibly sealed by one or two fastening mechanisms each having two parts which join to seal together a first side and a second side of the bag. Each fastening mechanism has a first part joined to or passing through the first side which is captured or adhesively bonded to a second part joined to or passing through the second side. The bag has indicia indicating where to cut to remove the locking mechanisms to access the port. The bag is sized such that the port cannot be rotated to a position adjacent the open end.

### CROSS REFERENCES TO RELATED APPLICATIONS

This application claims the benefit of priority of U.S. provisional App. No. 62/615,173, filed Jan. 9, 2018, the disclosure of which is incorporated by reference herein.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

The present invention relates to catheters which cooperate with lock-out devices to prevent unauthorized access by intravenous drug abusers.

Abusers of intravenous drugs such as heroin may suffer from medical conditions requiring insertion of catheters. Be it the treatment of infections by regular intravenous injection of antibiotics, or amelioration of kidney malfunctions by regular dialysis, the drug abuser may be provided with an intravenous Peripherally Inserted Central Catheter or PICC line or other catheter arrangement. This catheter, while usually inserted in the hospital, can remain in place for weeks or months, and makes it a simple matter to rapidly inject necessary fluids without difficulty in finding a vein. This ease of intravenous access, however, can be a serious hazard for outpatients who are habitual drug abusers, who can take advantage of this catheter to self-administer illicit narcotics or other controlled substances. Providing a drug abuser with an unsupervised catheter for outpatient care can lead to drug overdose and death. To protect these high-risk patients, who might otherwise not need full-time nursing care, they must be hospitalized and retained under supervision for as long as the catheter is in place. Hospitalisation is vastly more costly than outpatient care, and represents a less than optimal utilization of health care resources.

What is needed is an arrangement that can provide ready indication to supervisory medical personnel of abuse of a PICC line.

### SUMMARY OF THE INVENTION

A bag has an open side closeable with a two part seal or closure. The open side provides for the passage of a catheter port terminating a catheter line into the bag, allowing the port to be contained within the bag. The passageway formed between the two part closure for the catheter line is narrow such that the catheter port cannot be removed from the bag when the two part closures are sealed i.e., closed. Each part of the two part closure forms a one way, locking only seal, such that the catheter port cannot be removed without cutting one or both of the two parts of the seal. The seal can be a mechanical mechanism such as employed in a Ziplock® bag seal, only rather than being re-sealable, one side of the seal is captured in the other side of the seal and cannot be unsealed at all or at least without leaving clear evidence of the forced opening of the bag.

In another embodiment, the seal is formed by one or more continuous lines or rows of zip tie like fasteners where one or both structures deform as they are brought together and as so engaged the one or both deformed structures expand and can no longer be separated. The closure can be integral with the bag or can penetrate the bag or wrap the bag material about a structure in the closure so the bag side walls cannot be pulled out of the closure. In addition, the bag open side can be heat sealed or welded, e.g., ultrasonically, or adhesively bonded, particularly where a chemical reaction of the opposed sides of the bag opening forms an adhesive bond.

The bag is formed from a material of a type or thickness which is difficult to tear but can be cut with the proper scissors or sheared with a press which fits over the bag end and shears off the closures.

It is an object of the present invention to provide a catheter protective plastic bag which closes around a catheter port.

Further objects, features and advantages of the invention will be apparent from the following detailed description when taken in conjunction with the accompanying drawings.

### BRIEF DESCRIPTION OF THE DRAWINGS

FIG. 1 is a prospective view of the PICC guard of this invention formed by a bag and two bag closures.

FIG. 2A is a fragmentary cross-sectional view of the PICC guard of FIG. 1, taken along section line 2A-2A.

FIG. 2B is a fragmentary cross-sectional view of the PICC guard of FIG. 2A shown in the partially closed configuration.

FIG. 2C is a fragmentary cross-sectional view of an alternative embodiment PICC guard of the present invention, having a mating barb closure arrangement.

FIG. 2D is a fragmentary cross-sectional view of another alternative embodiment PICC guard of the present invention, having a two-part adhesive closure arrangement.

FIG. 2E is a fragmentary cross-sectional view of an alternative embodiment PICC guard of the present invention, having a barb engagement closure arrangement.

FIG. 3 is an illustrative view of two PICC guards of FIG. 1 shown as used with a patient.

FIG. 4 is a top plan view of an alternative embodiment PICC guard of this invention formed by a bag and a single bag closure.

FIG. 5 is an illustrative view of the bag of FIG. 1 shown open before the inlet port is placed in the bag and the bag is closed with devices for forming the bag closures.

### DESCRIPTION OF THE PREFERRED EMBODIMENTS

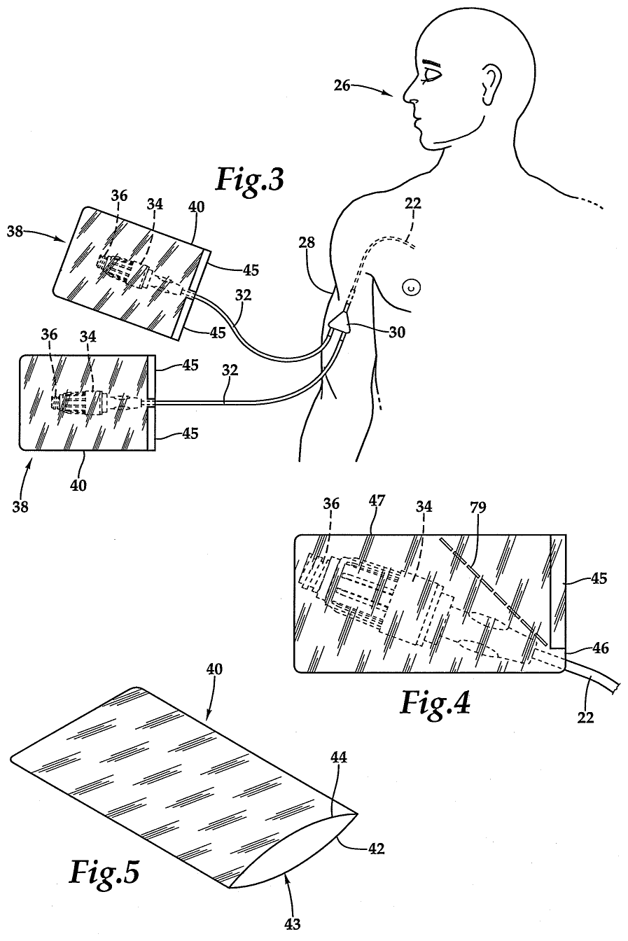

Referring more particularly to FIGS. 1-5, wherein like numbers refer to similar parts, a Peripherally Inserted Central Catheter (PICC) assembly 20 is shown in FIG. 3. The PICC assembly 20 has a flexible catheter 22 which is inserted into the upper extremity 24 of the patient 26, for example, an arm 28. The catheter assembly 20 will typically have a PICC line base 30 from which the catheter emerges and enters the patient through the skin. The PICC line base 30 is positioned to hold the catheter against movement by attachment to the patient, for example, by tape. One or more injection lines 32 extend from the PICC line base 30, each of which is terminated by an inlet port 34. The inlet ports 34 provide entryways to the injection lines 32 for delivery of fluids from an injection source such as a syringe or a total parenteral nutrition bag or whatever injectable the patient requires. As shown in FIG. 3, the inlet ports may be provided with an inlet side positive displacement connector 36, such as the MaxPlus® clear needleless connector made by Carefusion Corporation of San Diego, Calif., and such as disclosed in U.S. Pat. No. 5,730,418.

As shown in FIG. 1, a PICC guard 38 has a plastic and/or foil bag 40 which strongly resists puncture by a needle. As shown in FIG. 5, the bag 40 has a first side 42 and a second side 44 which define an opening 43 through which an inlet port 34 may be placed in the bag. The first and second sides 40, 42 are then joined to each other by two fastener mechanisms 45 which clamp the left and right sides of the bag opening leaving only a small opening 46 between the fastening mechanisms through which one or more injection lines 32 can pass out of the bag 40 while trapping the PICC inlet port 34. The bag is preferably non-square, as shown in FIG. 1, so the bag extends away from the entrance opening a given distance which is greater than the length of the port 34 to fully receive the port within the bag. The bag furthermore has a width perpendicular to the given distance which is less than the given distance, such that the port cannot be turned within the bag to present the port inlet side opening at the bag opening. The bag 40 is arranged to closely match the size of the inlet port 34 such that the port cannot be rotated within the bag such that the port inlet side 36 is accessible to a needle passing through the small opening 46 between the fastening mechanisms 45 through which the injection lines 32 pass.

Although two fastening mechanisms 45 are preferred, the use of a single fastening mechanism 45 is possible as shown in the embodiment of FIG. 4 by forming the bag 47 with the left or right side already sealed or by arranging the injection lines to pass out of the bag on one side of the opening and employing a single clamping mechanism 45 to reduce the bag opening to both constrain the rotation of the inlet port 34 and to form the small opening 46 on one side of the larger opening 43 through which the inlet port 34 is placed into the bag 40 as shown in FIG. 5.

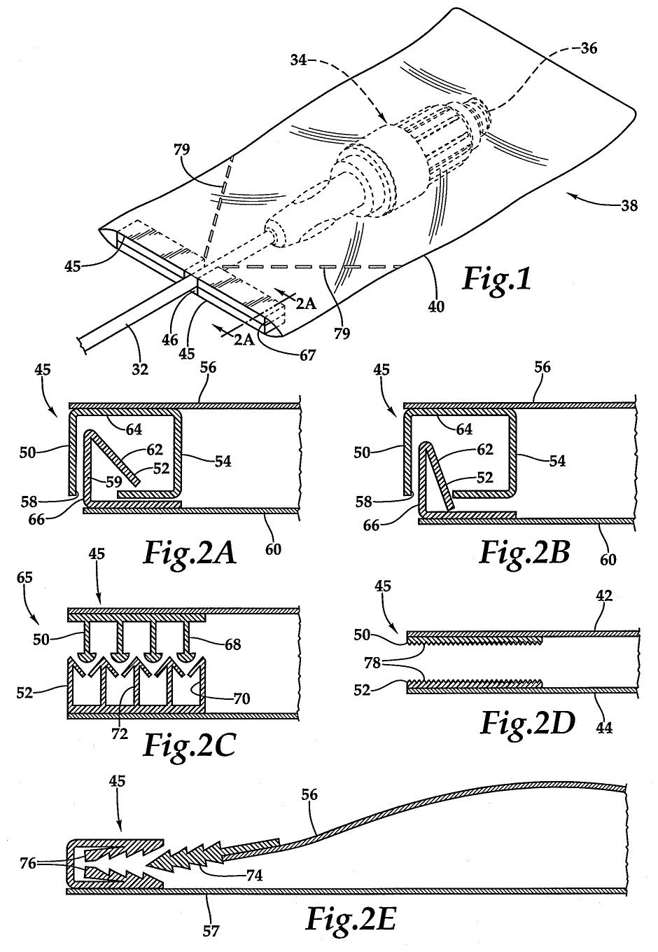

The one or two fastening mechanisms 45 may be a mechanical connection formed of a first part 50 which irreversibly locks to a second part 52. As shown in FIG. 2A the first part 50 may be comprised of a linear receptacle 54 which is attached along an interior portion of the bag side 56 forming the large opening 43. The linear receptacle 54 has a narrow linear opening 58. The second part 52 has a stiffened member 59 such as formed by a thick or angular portion which is attached along a second portion or second bag side 60. Extending from the stiffened member 59 is a flexible member 62 which must deflect as it passes through the narrow linear opening 58, as shown in FIG. 2B. The linear receptacle 54 has an internal volume 64 which connects to the narrow linear opening 58 such that the internal volume allows the flexible member 62 to return to its un-deflected shape once it passes through the narrow linear opening 58 as illustrated in FIG. 2A thereby locking the flexible member 62 within the linear receptacle 54 to irreversibly connect the first portion or side of the bag 56 to the second portion or side 60 of the bag 40. As shown in FIG. 1, the linear receptacle 50 portion of the closure mechanism 45 may be provided with closed ends 67 to prevent any access to the internal volume 64 except by way of the narrow linear opening 58 which is substantially blocked by portions 66 of the flexible member 62. The ends 67 comprise walls which extend perpendicular to the axis of the linear receptacle 50.

FIG. 2C shows an alternative embodiment fastening mechanism 65 comprised of one or more rows of fixed or flexible members which interpenetrate each other to form a reversible lock. The first part of the closure attached to one side of the bag has a plurality of mushroom shaped rigid members 68 which pass into narrow gaps 70 defined between arrow-like flexible members 72 forming a second part of the closure attached to the other side of the bag. When the members extend between the flexible members they are locked on one side of the flexible members. The rigid members 68 extend perpendicular to the first bag side, and the flexible members 72 comprise a plurality of capture members extending perpendicular to the second bag side and towards the locking mechanism first part members such that when the first side of the bag is pressed towards the second side of the bag the plurality of rigid members pass between the plurality of flexible capture members 72 and are captured therein.

FIG. 2E shows a ratchet connection similar to that used in cable ties where a single or double sided ratchet 74 is attached to the bag side 56 and slides between counter ratchet surfaced members 76 flexibly extending within a linear receptacle 77 which is attached to the other bag side 57. The ratchet 74 is insertable into the linear receptacle 77, but is then not removable therefrom.

FIG. 2D employs layers 78 of a one or two part adhesive on opposed bag sides 42 and 44 which form an irreversible seal when pressed together. The layers 78 of adhesive may be covered by release strips (not shown) which overlie the adhesive layers to prevent premature joining of the layers, and are formed of a material which, while retained on the adhesive layers 78, may be easily stripped away to permit joining of the adhesive layers.

As shown in FIG. 1, the PICC guard 38 bag 40 may be provided with indicia 79, e.g., dotted lines, which indicate where to cut to open the bag 40 so the inlet port 34 can be removed from the bag.

It should be understood that the bag may be formed of any suitable material which will resist needle punctures or which will readily show the needle punctures such as by an interaction of two or more layers of the bag in such a way as to change the appearance of the bag around the needle puncture such that the puncture is readily visible.

ASTM F2878-10 Standard Test Method for Protective Clothing Material Resistance to Hypodermic Needle Puncture may be used to test materials for resistance to 21, 25 and 28 gauge needles to a selected level of puncture resistance which can be used to specify the material used in forming the bags 40. It should be understood that the bags may be formed of plastics incorporating high strength fibers such as carbon fibers, glass fibers, ceramic fibers, metal fibers and the fibers may be either woven or matted. It should be understood that in some cases it may be necessary to use a punch or shearing device which can be slipped over the locked bag opening and will allow greater cutting force than can readily be applied by scissors to sever the connectors from the bag. Alternatively, some type of tool or mechanism can be used to unlock or break the locking mechanisms 45.

It is understood that the invention is not limited to the particular construction and arrangement of parts herein illustrated and described, but embraces all such modified forms thereof as come within the scope of the following claims.

I claim:

1. A catheter tamper evident locking device comprising:
a bag, having an open end, the bag being comprised of a material which is resistant to needle puncture;
at least one locking mechanism arranged to close the bag open end such that a flexible catheter attached to an injection port can pass through the open end while the injection port retained within the bag is prevented from passing through the open end; and
wherein the bag has dimensions such that the inlet port placed in the bag, such that when the inlet port extends from the flexible catheter away from the bag open end, the inlet port cannot be rotated within the bag such that the inlet port doubles back along the flexible catheter so as to be accessible to a hypodermic needle passing into the open end of the bag along the flexible catheter.

2. The catheter tamper evident locking device of claim 1 wherein the at least one locking mechanism is comprised of a first locking mechanism, and a second locking mechanism which are arranged to close the open end and form an opening from a portion of the open end which is between the first locking mechanism and the second locking mechanism.

3. The catheter tamper evident locking device of claim 1 wherein the bag has a first wall and a second wall which together define the open end;
wherein the at least one locking mechanism comprises a first part connected to the first wall adjacent to the open end, and a second part connected to the second wall adjacent to the open end.

4. The catheter tamper evident locking device of claim 3 wherein the at least one locking mechanism first part comprises a linear receptacle extending along the first wall which has a linear opening into a linearly extending volume;
wherein the locking mechanism second part extends along the second wall and has a linearly extending member and attached thereto a flexible linearly extending member which is collapsible towards the linear extending member such that the linear extending member and the flexible linearly extending member can pass through the linear opening into the linearly extending volume, and the linearly extending volume is such that the flexible linear extending member is free to move away from the linearly extending member such that the first part and the second part of the at least one locking mechanism are locked together; and
wherein the linear receptacle has end walls bracketing the linearly extending volume such that the volume can be accessed only through the linear opening into the linearly extending volume.

5. The catheter tamper evident locking device of claim 3 wherein the locking mechanism first part is an adhesive layer on the first wall, and the locking mechanism second part is an adhesive layer on the second wall.

6. The catheter tamper evident locking device of claim 3 wherein the locking mechanism first part has at least one ratchet surface parallel to the first wall, and the locking mechanism second part has a receptacle forming an opening which is parallel to the second wall, the opening having a counter ratcheting surface into which the locking mechanism first part can be slid to be locked within said receptacle.

7. The catheter tamper evident locking device of claim 3 wherein the locking mechanism first part has a plurality of rigid members which extend perpendicular to the first bag side, and wherein the locking mechanism second part has a plurality of flexible members extending perpendicular to the second bag side and towards the locking mechanism first part plurality of rigid members such that when the first side of the bag is pressed towards the second side of the bag the plurality of rigid members pass between the plurality of flexible members and are captured therein.

8. A device for evidencing tampering with a Peripherally Inserted Central Catheter (PICC) assembly having a port connected to a flexible catheter, the device comprising:
a flexible bag having a first wall overlying a second wall, an entrance opening being defined between the first wall and the second wall and an interior volume being defined between the first wall and the second wall;
a closure having portions mounted to the first wall and the second wall, such that in an open configuration, a PICC port can be passed into the interior volume of the bag with its connected flexible catheter extending out of the entrance opening, and in a second closed configuration in which closure portions mounted to the first wall and the second wall of the bag are engaged with each other in a secure fashion which cannot be separated without giving evidence of tampering, and wherein in the second closed configuration there is a narrow opening into the bag defined adjacent the closure through which the connected flexible catheter may freely exit, while restricting the exit of the connected PICC port.

9. The device of claim 8 wherein the closure comprises:
a first part connected to the first wall adjacent to the entrance opening; and
a second part connected to the second wall adjacent to the entrance opening, the second part having portions which non-releasably connect to the first part when the two parts are pressed against one another.

10. The device of claim 9 wherein the closure first part comprises a linear receptacle extending along the first wall which has a linear opening into a linearly extending volume;
wherein the closure second part extends along the second wall and has a linearly extending member and attached thereto a flexible linearly extending member which is collapsible towards the linear extending member such that the linear extending member and the flexible linearly extending member can pass through the linear opening into the linearly extending volume, and the linearly extending volume is such that the flexible linear extending member is free to move away from the linearly extending member such that the first part and the second part of the at least one locking mechanism are locked together.

11. A tamper evident catheter assembly comprising:
a flexible catheter for insertion within a patient to deliver fluids thereto;
a port connected to the catheter and capable of receiving a needle therein for reception of injectable fluids, the port extending a first distance away from the catheter and terminating at a port inlet side opening;
a flexible puncture-resistant bag having a first wall overlying a second wall, an entrance opening being defined between the first wall and the second wall and an interior volume being defined between the first wall and the second wall, the bag extending away from the entrance opening a second distance greater than the first distance to fully receive the port within the bag, the bag having a width perpendicular to the second distance which is less than the first distance, such that the port cannot be turned within the bag to present the port inlet side opening at the bag opening; and
a closure having portions mounted to the first wall and the second wall, the closure portions mounted to the first wall and the second wall of the bag are engaged with each other in a secure fashion which cannot be separated without giving evidence of tampering, and wherein a narrow opening into the bag is defined adjacent the closure through which the connected flexible catheter may freely exit, while restricting the exit of the connected port.

12. The tamper evident catheter assembly of claim 11 wherein the closure comprises:
a first part connected to the first wall adjacent to the entrance opening; and
a second part connected to the second wall adjacent to the entrance opening, the second part having portions which non-releasably connect to the first part when the two parts are pressed against one another.

13. The tamper evident catheter assembly of claim 12 wherein the closure first part comprises a linear receptacle extending along the first wall which has a linear opening into a linearly extending volume; and
wherein the closure second part extends along the second wall and has a linearly extending member and attached thereto a flexible linearly extending member which is collapsible towards the linear extending member such that the linearly extending member and the flexible linearly extending member can pass through the linear opening into the linearly extending volume of the linear receptacle, and the linearly extending volume is such that the flexible linearly extending member is free to move away from the linearly extending member such that the first part and the second part of the at least one locking mechanism are locked together.

14. The tamper evident catheter assembly of claim 13 wherein the linear receptacle has end walls bracketing the linearly extending volume such that the volume can be accessed only through the linear opening into the linearly extending volume.

15. The tamper evident catheter assembly of claim 12 wherein the locking mechanism first part is an adhesive layer on the first wall, and the locking mechanism second part is an adhesive layer on the second wall.

16. The tamper evident catheter assembly of claim 12 wherein the locking mechanism first part has at least one ratchet surface parallel to the first wall, and the locking mechanism second part has a receptacle forming an opening which is parallel to the second wall, the opening having a counter ratcheting surface into which the locking mechanism first part can be slid to be locked within said receptacle.

17. The catheter tamper evident locking device of claim 12 wherein the locking mechanism first part has a plurality of rigid members which extend perpendicular to the first bag side, and wherein the locking mechanism second part has a plurality of flexible capture members extending perpendicular to the second bag side and towards the locking mechanism first part plurality of rigid members such that when the first side of the bag is pressed towards the second side of the bag the plurality of rigid members pass between the plurality of flexible capture members and are captured therein.
